(19) United States
(12) Patent Application Publication
(10) Pub. No.: US 2011/0160607 A1
(43) Pub. Date: Jun. 30, 2011

(54) QEED-Guided Selection and Titration of Psychotropic Medications

(75) Inventors: Erwin R. John, Mamaroneck, NY (US); Leslie S. Prichep, Mamaroneck, NY (US)

(73) Assignee: NEW YORK UNIVERSITY, New York, NY (US)

(21) Appl. No.: 12/744,619

(22) PCT Filed: Dec. 12, 2008
(86) PCT No.: PCT/US08/86609

§ 371 Date: Jan. 14, 2011

Related U.S. Application Data
(60) Provisional application No. 61/014,618, filed on Dec. 18, 2007.

(51) Int. Cl. A61B 5/0476

(52) U.S. Cl. 600/544

(57) ABSTRACT

Data corresponding to brain electrical activity of a patient from a QEEG is processed to determine which brain activity data deviates from that of a normative profile. The deviant brain activity data is used to determine a brain state vector (BSV) in a multidimensional brain electrical signal space, wherein the orientation of the BSV indicates the nature of the deviation from a normal state and may be used to prescribe a medicine to the patient and wherein the length of the BSV quantifies the degree of abnormality exhibited by the patient and may indicate the dosage of the medicine to be prescribed to the patient.

### PRIORITY CLAIM

This application claims priority to U.S. Provisional Application Ser. No. 61/014,068, entitled “QEEG-Guided Selection and Titration of Psychotropic Medications” filed on Dec. 18, 2007. The specification of the above-identified application is incorporated herewith by reference.

### FIELD OF THE INVENTION

The present invention relates to a method and system for managing pharmacological patient treatment based on measurements of the electrical activity of the brain.

### BACKGROUND INFORMATION

The treatment of developmental, neurological, or psychiatric disorders may involve prescribing one or more medications. In selecting the medication and determining a dosage, a physician typically performs a symptomatological diagnosis that is normally compliant with a formal schedule of diagnostic criteria. In performing such a diagnosis, the physician will determine the symptoms, either by observing the patient for abnormal behavior or listening to the patient describe his symptoms. After evaluating the symptoms in light of clinical intuition and past experience, the physician may prescribe one or more medications.

Because of its subjective nature, this typical approach to prescribing medications can be inaccurate. If a diagnosis lacks an objective basis rooted in physio-neurological measurements, physicians' assessments may be far from the mark, causing the prescription of medications that are not beneficial or even harmful.

### SUMMARY OF INVENTION

In an exemplary embodiment of the present invention, data corresponding to the electrical activity produced by the brain of a patient such as, for example, the data of a quantitative electroencephalogram (QEEG), is processed to determine which brain activity data for the patient deviates from corresponding brain activity data of a normative profile. The deviant brain activity data is used to determine a brain state vector (BSV) in a multidimensional brain electrical signal space. The orientation of the vector indicates the nature of the deviation from a normal state and may also be used to select the medicine that ought to be prescribed to the patient, while the length of the vector quantifies the degree of abnormality exhibited by the patient and may also indicate the dosage to be administered to the patient.

### BRIEF DESCRIPTION OF DRAWINGS

FIG. 1 shows an exemplary embodiment of a system for determining a pharmacological treatment for a patient based on measured electrical brain activity.

FIG. 2 shows a flow diagram showing an operation of the system of FIG. 1 in determining a treatment for a patient.

FIG. 3 shows a flow diagram relating to a method for determining a brain state vector (BSV) for a patient.

FIG. 4 shows an exemplary BSV having a length and orientation exhibiting an abnormal brain functioning.

FIG. 5 shows a flow diagram relating to a method by which the system of FIG. 1 uses a medicine database 5 to select a medicine and dosage for treating a patient.

FIG. 6 shows an exemplary BSV drug diagnosis method for treating a patient.

FIG. 7 shows a flow diagram relating to a method for creating an MRS database for diagnosing and treating a patient.

### DETAILED DESCRIPTION

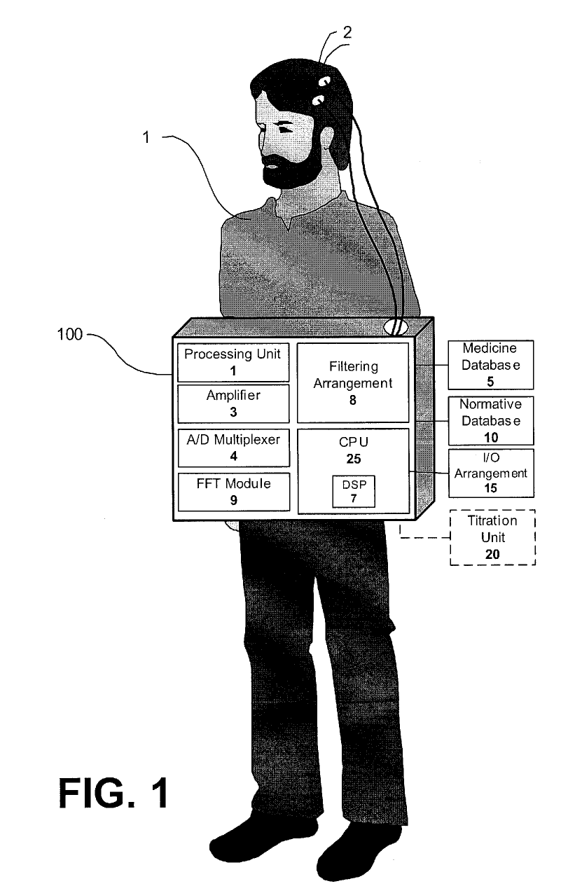

FIG. 1 shows an exemplary embodiment of a system 100 for managing the pharmacological treatment of a patient 1. Referred to herein as a QEEG-guided selection and titration of medication (QGSM) system 100, the exemplary system of FIG. 1 (i) collects and analyzes EEG (electroencephalogram) information from an array of electrodes 2 applied to the scalp of patient 1, (ii) constructs and stores a BSV to represent in signal space abnormal brain function of patient 1, (iii) examines a medicine database 5 to identify a drug or combination of drugs that would best alter the BSV to a normal state, and (iv) monitors and quantifies the changes in the BSV as the drug regimen suggested by the invention is administered with the purpose of identifying a drug or drug combination and dosages that minimizes the magnitude of the BSV without rotating its direction, which would reflect undesirable side effects.

In the system 100 of FIG. 1, a plurality of EEG electrodes 2 (e.g., 19-21 electrodes) are removably secured to the scalp of the patient 1 in accordance with the International 10/20 Electrode Placement System, as would be understood by those of skill in the art. Additional removable electrodes may be utilized as desired while additional reference electrodes (unilateral or linked) may be removably positioned on the mastoids or earlobes (A1, A2). Electrooculogram (BOG) electrodes may optionally be placed at an outer canthus of the eye to facilitate artifact rejection. As would further be understood by those of skill in the art, electrodes may also be placed on the central vertex (Cz) to record brainstem potentials and on the cheekbone to serve as a ground.

Alternatively, a subset of the number of electrodes prescribed by the 10/20 Electrode Placement System may be applied to the patient 1. Specifically, in one example, the electrodes may be applied only to the forehead such that each of the electrodes is only sensitive to activity in the frontal regions of the brain. Knowledge of the normative covariance matrix describing relationships between such a subset and the full 10/20 array may be used to augment the data recorded directly from the subset. The reduced number of electrodes is less cumbersome to the person applying the embodiment of the present invention and may be particularly useful for a portable version of the embodiment of FIG. 1. With a reduced number of electrodes, this portable implementation may be used, for instance, by EMT personnel who must quickly assess at the scene whether an individual is suffering from a cerebral disorder. For example, the subset of electrodes may be positioned on an easily mounted headband or hat over the forehead so that good skin/electrode contact may be made without attending to the removal of hair, etc.

In the stationary implementation of the QGSM system 100, the electrodes 2 preferably use a standard electrolyte gel, or other application method, so that the impedance of each electrode-skin contact is below 5000 ohms. Alternatively, for some applications, a plurality of needle electrodes, a pre-gelled electrode appliance with adhesive or other means of fixation, or an electrode cap or net with preselected electrode positions may be used. The QGSM system 100 automatically checks the electrode-skin impedance of each electrode 2 at frequent intervals, (e.g., every minute), and displays a warning (e.g., a red LED light) if any such impedance increases above 5000 ohms.

Electrode leads connect each of the electrodes 2 to a respective EEG/EP amplifier 3 of a processing unit 1, with each amplifier 3 preferably including an input isolation switch (e.g., a photo-diode and LED coupler) to prevent current leakage to the patient 1. The amplifiers 3 are high-gain, low-noise amplifiers, preferably having, for example, a maximum peak-to-peak noise of 1 microvolt, a frequency range of 0.5-200 Hz, a fixed gain of 10,000 and a common mode rejection of 100 dB or more (4 amplifiers). The amplifiers 3 are analog amplifiers and may be connected to an analog-to-digital multiplexer 4 (“A/D multiplexer”). Alternatively, the amplifiers 3 may be digital 24-bit amplifiers, thus obviating the need for the A/D multiplexer 4. In the case where amplifiers 3 are analog, the A/D multiplexer 4 may sample the amplified analog brain waves at a rate of, for example, 5 KHz for each channel. The A/D multiplexer 4 is connected to a filtering arrangement 8 which is, in turn, connected to a central processing unit 25 including a dedicated digital signal processor (“DSP”) 7, such as, for example, model TMS320C44® (Texas Instruments). Alternatively, the DSP 7 may be a Pentium 4 Processor® (Intel) or a digital signal processor such as the TMS320C44® (Texas Instruments) along with a microprocessor.

Using techniques that would be understood by those skilled in the art, the system 100 collects analog EEG signals from the patient 1, digitizes the signals via the A/D multiplexer 4 and, under the control of a suitably programmed DSP 7 in CPU 25, performs on the digitized EEG signals a Fast Fourier Transform via FFT module 9 to extract from the EEG signals QEEG data representing the power spectra of the EEG signals at predetermined frequency intervals. The present invention is also consistent with QEEG data that has been derived from techniques other than FFT, such as a Wavelet Transform Analysis, Independent Component Analysis, etc. As shall be explained more fully below, the system 100 according to the present invention constructs from the QEEG information a brain state vector (“BSV”), which may be used to optimize pharmacological treatment of the patient 1.

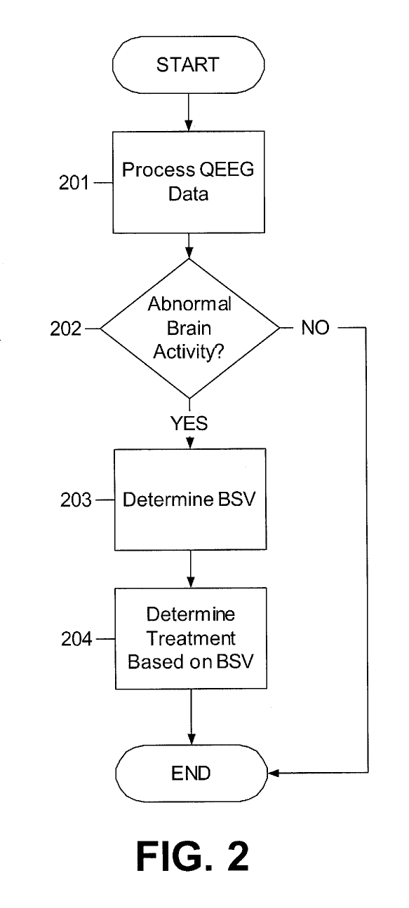

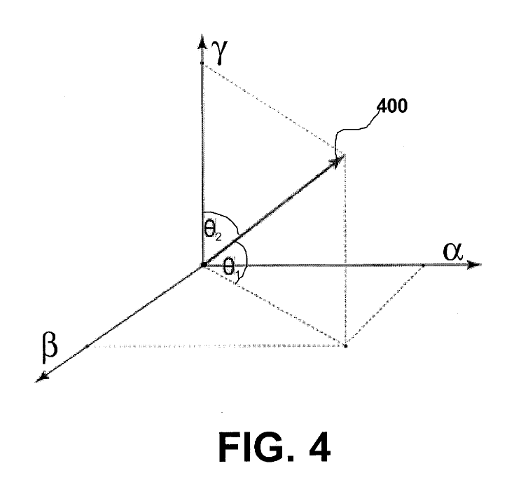

FIG. 2 illustrates a flow diagram showing the operation of the system 100 of FIG. 1. According to FIG. 2, after QEEG data has been generated based on the above description, the CPU 25 processes the QEEG data (step 201) to determine whether the patient is exhibiting abnormal brain activity (step 202), This determination involves comparing the QEEG data of the patient to normative QEEG data stored in normative database 10. The values maintained in normative database 10 may, for instance, represent normal brain activity data for a control population of persons across a wide spread of ages (e.g., ages newborn to 90) or may include a self-normative data set established while the patient 1 is exhibiting substantially normal brain functioning. If the CPU 25 determines that the QEEG data for patient 1 exhibits abnormal brain activity, the CPU 25 performs the method of FIG. 4 (described below) to determine a BSV for that patient 1 (step 203). The BSV may be represented as a Mahalanobis Distance across a set of standard scores or Z-scores, correcting for the intercorrelations between or among any of the QEEG descriptors. According to the exemplary embodiment, the BSV represents, within a multidimensional brain electrical signal space, the extent to which the QEEG data determined by the CPU 25 deviates from corresponding values from the normative QEEG data stored in normative database 10. After a BSV has been determined for patient 1, the CPU 25 determines a treatment for patient 1 using the parameters of the BSV to select a treatment from the medicine database 5 (step 204). This selection is described in further detail below.

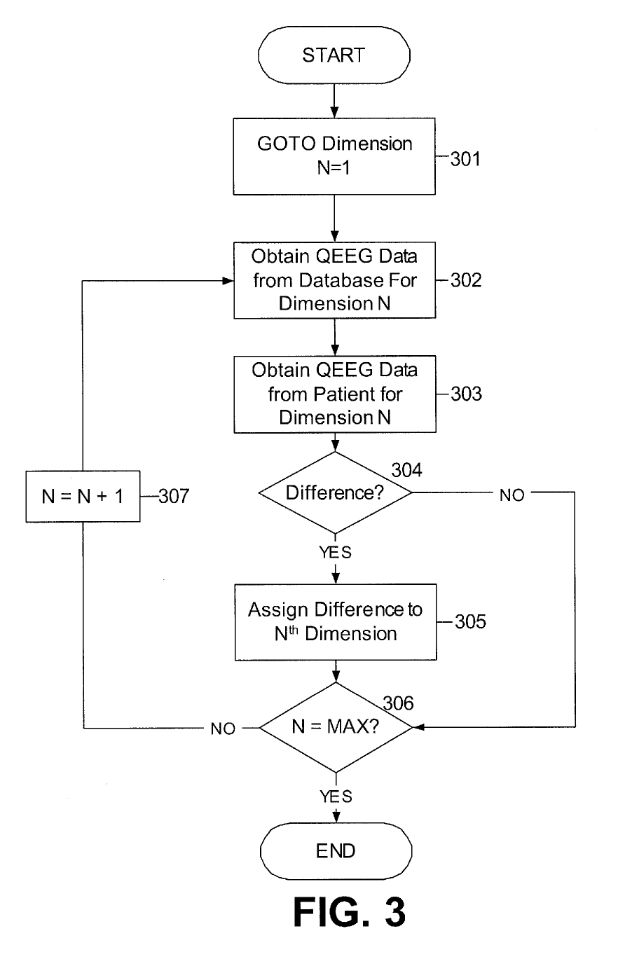

FIG. 3 is a flow diagram illustrating a method for constructing a BSV according to the present invention. In this exemplary embodiment, a BSV is an n-dimensional vector the length of which quantifies a degree of abnormality in brain activity while its orientation signifies a particular medicine to be administered to the patient 1. As an empirical matter, distinctive BSVs have been described for different developmental, neurological and psychiatric disorders, and different classes of psychotropic drugs have been observed to induce BSVs in a control population of normal persons (i.e., persons for whom no BSV can be generated when not under the influence of such drugs because they are not suffering from any cerebral disorder or brain injury). As shall be explained in more detail below, each BSV derived from the control population exhibits an orientation that is distinctive for a particular class of drugs. This relationship between vector orientation and drug identity permits a patient 1 to be treated by having his BSV determined and compared to the BSVs derived from the control population.

Each dimension n of the BSV may, for instance, signify a single frequency band of interest while the value assigned to each dimension n signifies a deviation between the brain activity measured from the patient 1 (e.g., in the QEEG data) for the frequency band of interest and the normative brain activity for that frequency band obtained from normative database 10. The elements comprising the BSV may also represent symmetries or synchronies between spectral descriptors in selected regions or sets of regions. The system 100 may calculate such deviations for each of a plurality of frequency bands or sets of descriptors. For instance, the system 100 may determine whether deviations in QEEG data exist for any of the alpha (8-14 Hz), beta (14-30 Hz), gamma (26-100 Hz), delta (0.5-4 Hz), and theta (4-8 Hz) frequency bands in any electrode or the ratio of voltages or the phase relationship of oscillations at any frequencies within or between any pair of electrodes. Other frequency bands of interest may serve as the basis for the analysis performed by system 100. For example, the system 100 may perform the analysis on frequency intervals located within the very narrow band (VNB) power spectrum. As mentioned above, each dimension compressed into the BSV may correspond to one frequency band. If the frequency bands of interest are the alpha, beta, gamma, delta and theta bands, then the BSV will be compressed from four dimensions, representing the standard score or Z-value for each band. The present invention is not limited to constructing BSVs composed of dimensions associated with the alpha, beta, gamma, delta, and theta bands, but is instead capable of constructing BSVs having as many dimensions as there are frequency bands of interest, relations or interactions among sensors of brain activity in a particular application, or other descriptors (e.g., blood pressure, heart rate, EKG). If the user of the system 100 is interested in determining the deviation from the norm population in QEEG data in 12 frequency bands, then system 100 is capable of computing a brain state vector from data with twelve dimensions, one for each frequency band of interest.

In order to perform the analysis on selected frequency bands, processing unit 1 includes a filtering arrangement 8 that operates according to known high-pass, low-pass and band-pass filtering techniques in order to isolate QEEG data for specific frequency intervals of interest. For example, known filtering techniques may be employed such as those described in U.S. Pat. No. 4,705,049 to John and U.S. Pat. No. 6,556,861 to Prichep the entire disclosures of which are hereby incorporated by reference in their entireties.

The method of FIG. 3 begins by selecting one of the n-dimensions for evaluation (step 301). The selection of which dimension n to start with may be done arbitrarily by determining a threshold for the statistical significance of all measurements to be considered for incorporation into a BSV. For the sake of simplicity, the illustrative example discussed here for FIG. 3 shall be limited to just those values derived from measures for the alpha, beta and gamma frequency bands (see the exemplary BSV vector of FIG. 4, discussed below). Notwithstanding the example discussed herein, the BSVs determined by the present invention may encompass as many dimensions as there are frequency bands of interest, or other descriptors of interest, with each N value correlating to a frequency band of interest. For instance, instead of brain electrical activity, the system of FIG. 1 may instead rely on statistically significant differences in measures relating to blood flow or metabolic activity in the brain, EKG descriptors, and incorporate such measures into a Mahalanobis distance if these measures were included in a multimodal or crossmodal covariance matrix. Alternatively, measurements such as cerebral blood flow measures from SPECT or regional glucose metabolic measures from different brain region obtained by PET may be used. It is desirable that all measurements to be computed by a BSV be resealed so as to have the same dimensionality, preferably probability of deviation from a reference or normative value expressed as a standard deviation. As stated above, this computation or transformation is done using well-known mathematical techniques (e.g., Mahalanobis distance technique), the purpose of which is to rescale the measurements to a form exhibiting Gaussianity. Thus, in the exemplary embodiment, the physical measurement (whether of brain electrical activity, cerebral blood flow or metabolic activity) is resealed and expressed in units of standard deviation.

CPU 25 obtains the QEEG value for all Z-scores in the set of descriptors which are above the selected threshold relative to a normative database 10 (step 302). The QEEG value derived from patient 1 used in this method can be a value that has just been calculated from a real-time EEG obtained while patient 1 is still joined to the system via the scalp electrodes 2, or it can be one that was previously calculated and recorded onto an electronic storage medium (e.g., flash memory, CD-ROM, etc.) and read out for the purpose of performing the analysis described herein (step 303). CPU 25 then determines whether a significant difference is present in the patient 1 relative to the normative database 10 (step 304). If there is a difference between the normative value and the present reading, the method proceeds to step 305 wherein the BSV may be conceptualized as a vector from the point of origin of the signal space and a tip at the multivariate distance from the origin, which would represent the mean values of the normative distribution of all the variables in the BSV.

If no difference is observed, the method may proceed to step 306, wherein the system 100 may determine if the method has reached the last dimension of interest. If the present N value is the last dimension of interest, the method may end. If the present dimension is not the last dimension of interest, the method may process to step 307 wherein a different N value, which corresponds to an EEG frequency band of interest, may be selected. In the exemplary embodiment shown, the system 100 scans for abnormalities in the EEG systematically based on the frequency band associated with each N value. Those skilled in the art will understand that the assignment of N values may be preset or may be defined by a user of the system 100 so as to limit or expand a scan set to a desired set of frequencies. After a new frequency band of interest has been assigned in step 307, the method returns to step 302 to scan this N value for abnormalities. After scanning of all frequency bands of interest has completed, the method may end and the resultant BSV computed.

As seen in FIG. 4, the exemplary BSV discussed above is a BSV 400 emanating from the origin of the multidimensional brain electrical signal space, herein illustrated for the sake of simplicity as a three-dimensional space as a function of the EEG frequency bands alpha (α), beta (β) and gamma (γ), with a magnitude of the BSV 400 indicated by its length and an orientation indicated by its angles of inclination (angle θ₁ and angle θ₂) with respect to the axes.

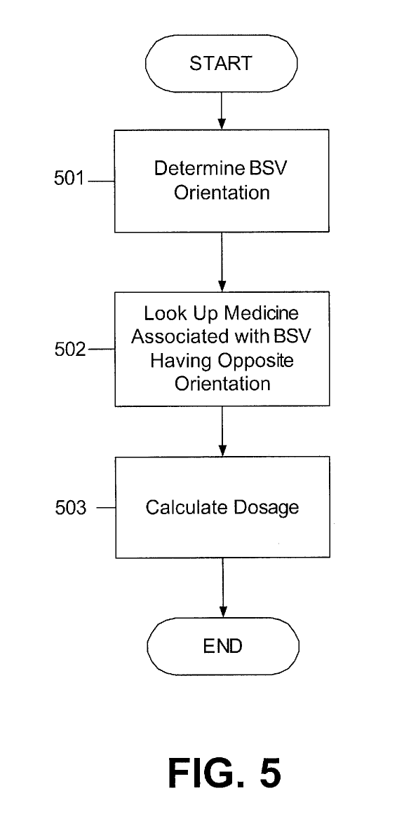

FIG. 5 shows a method for using a BSV to determine an optimal pharmacological treatment for a patient 1 from whom the BSV has been derived, The optimal treatment for the patient 1 is constructed by (1) determining a patient BSV for the patient 1 representing a magnitude and orientation of the deviation; (2) selecting from the medicine database 5 a drug associated with a BSV having an orientation most nearly opposite that of the patient BSV; and (3) determining a dosage of the selected drug based on the magnitude of the of the patient BSV. The notion of administering to the patient 1 a drug associated with a BSV having an orientation opposite that of the patient BSV is based empirically on the notion that in the multidimensional brain electrical signal space, a person exhibiting no symptoms indicative of brain disorder produces no BSV as the brain activity (or other quantitative descriptor such as blood flow) represents no significant deviation from the data of the normative database 10 over each of the dimensions of that vector space associated with the disorder. By applying to the patient 1 (who has produced a BSV indicative of this disorder) a medicine associated with an oppositely oriented BSV, the intent is to alter the BSV of the patient 1 so that it shrinks back toward the point of origin, with the return of normal brain electrical activity being accompanied by a corresponding decrease in the symptoms of the disorder.

In order to determine the association between a BSV orientation and particular medicines, a population of normal persons across a wide range of ages (e.g., ranging from newborn to 90 years) exhibiting no signs symptomatic of brain disorder would be prescribed various psychotropic drugs. The brain activity of each subject would be monitored before and after the administration of the drug to determine a BSV resulting from the drug with a magnitude of the resulting BSV being correlated to the dosage administered. For instance, the orientation and magnitude of each of the BSVs produced in a population by a drug would be correlated to the age and dosage for each subject. This data may then be recorded into the medicine database 5. Different drugs may be tested on the control population and the respective BSVs may be recorded until a complete medicine database 5 is produced, containing a plurality of drugs and respective BSV orientation/magnitude data correlated again by age of the subjects (age regression may be used).

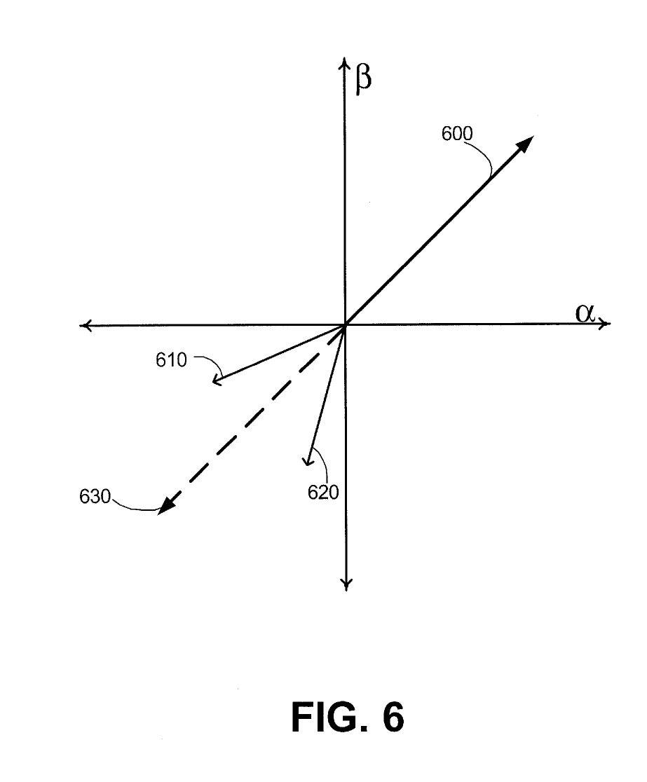

After determining the orientation of the BSV for patient 1 (step 501), the CPU 25 may use the orientation for the determined BSV to look up in the medicine database 5 the recommended pharmacological treatment for the patient 1. The medicine database 5 may be arranged as a look-up table, although any other data structure may be used that is capable of associating the determined orientation with a recommended treatment. Each BSV orientation may be associated with one or more drugs, drug regimens or class of drugs. In the case of a patient 1 exhibiting the BSV 600 shown in FIG. 6, as explained in further detail below, the appropriate drugs would be such that, when their BSVs are added together, the resultant vector may have a substantially opposite orientation to the BSV 600. After finding the opposite BSV in database 5, CPU 25 may locate the medication associated with this BSV (step 502). The CPU 25 may then use vector algebraic summation to calculate a recommended dosage of the medication based on the magnitude of the patient BSV (i.e., to obtain a magnitude of the opposite BSV substantially equivalent to that of the patient BSV).

The system shown in FIG. 1 outputs the recommended medicine and dosage via I/O arrangement 15, which may include, for example, a display enabling a doctor to read the recommended medication and suggested dosage. Alternatively, for example when managing a patient in an intensive care unit, the CPU 25 may administer the medication in an automated fashion by sending a signal to control any suitable automated titration unit 20 supplied with the medicines in medicine database 5 and connected to the patient 1 via an intravenous catheter or any other suitable drug delivery apparatus.

As the drug is administered to the patient 1 by the system 100, the system 100 may also monitor the patient BSV in real time to determine whether or when it is being reduced in length in a direction toward the point of origin of the multidimensional brain electrical signal space. The response of the patient 1 to the drug may be nonlinear, which means that after a certain point, a further increase in dosage will not produce a proportionate improvement in the patient 1, and may in fact begin eroding the previous benefits achieved at lower dosages. This monitoring may be done, for instance, by repeating at predetermined intervals the method of constructing a BSV to observe whether, over time, the BSV for the patient 1 is shrinking toward the point of origin. If increased dosages of the administered drug cause the patient 1 to regress, a real-time monitoring of the patient's BSV will indicate either that the BSV is no longer moving back to the point of origin of the vector space, or that the BSV is actually increasing in magnitude. At this point, a switch in medication is warranted. The system 100 may select another drug within the class of drugs associated with the orientation of the patient BSV. On the other hand, there may be no other drugs in the medicine database 5 that may have, or may be close to having, an orientation that is the opposite of the patient BSV orientation. In this case, the system 100 may need to select a combination of drugs associated with different respective orientations. The system 100 may select drugs with respective BSV orientations so that, when their respective BSVs are added through typical vector summation techniques, the summation will produce a resultant vector with an orientation that is the opposite (or close to the opposite) of the patient BSV.

FIG. 6 details an exemplary embodiment of the drug diagnosis technique of the present invention. In this exemplary embodiment, a patient 1 may exhibit a BSV 600. The system 100 may then determine that a combination of two drugs with respective BSVs 610 and 620 will, through vector summation, yield a resultant BSV 630, which is of the same magnitude but opposite direction of the BSV 600 of the patient 1. Those skilled in the art will understand that administering this combination of drugs will cause the BSV 600 of patient 1 to shrink back toward the point of origin. The drug combination can continue to be applied in this manner until the patient BSV 600 has been reduced back to the point of origin, or until a monitoring of the patient BSV 600 reveals that further dosages of the drug combination is not shrinking the BSV 600 or is actually causing it to increase in magnitude, at which point a new drug or drug combination is selected. This process can be repeated until the desired shrinkage of the patient BSV 600 is observed. Another aspect of the patient BSV 600 that may be monitored after administration of a selected drug regimen is whether the BSV 600 is rotating its direction (i.e., rotating away from the point of origin) thereby increasing the likelihood of side effects.

The exemplary system and method of the present invention may not be limited to use with the EEG and QEEG but may also be utilized to create BSVs for metabolic measures of different brain regions or for any other physiological measurements such as blood pressure, heart rate, electrocardiogram descriptors, cerebral blood flow measurements obtained from single photon emission computed tomography (“SPECT”), regional glucose metabolic measurements obtained from positron emission tomography (“PET”), etc.

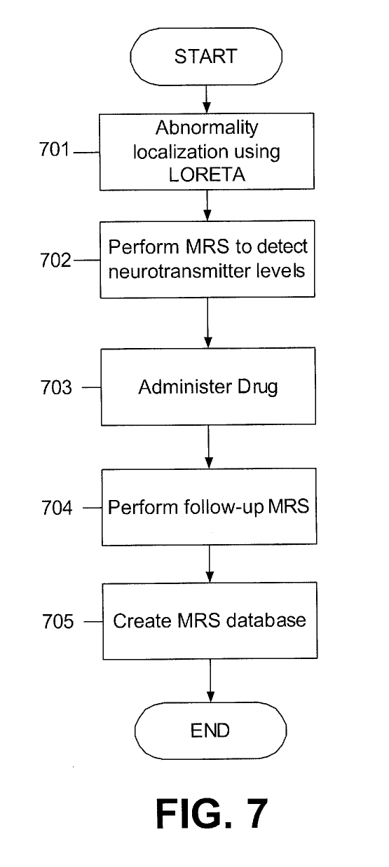

An exemplary alternate embodiment of the present invention is described with respect to FIG. 7, wherein Low Resolution Brain Electromagnetic Tomography (LORETA) may be used to identify regions of interest (ROI) in the brain, as those skilled in the art will understand (step 701). The LORETA technique may provide a three dimensional tomography of brain electrical activity, wherein the brain electrical activity may indicate a potential source of the patient's 20 pathology. Specifically, the LORETA technique may provide source localization of abnormalities in the brain, wherein the abnormality may be one or both of a multi-region abnormality and a multi-frequency abnormality, as those skilled in the art will understand. Magnetic resonance spectroscopy (“MRS”) may then be used to detect the levels of one or more specific neurotransmitters in the ROI (step 702). The MRS technique may be useful in that it may provide the spectral characteristics corresponding to precursors and/or metabolites to the neurotransmitters and/or electrolytes in the ROI.

An appropriate drug or combination of drugs may then be administered to the patient 1 (step 703). After the desired drug or combination of drugs has been administered to the patient 1, a follow-up MRS may be performed to determine the level of efficacy of the drug(s) (step 704). Specifically, the follow-up MRS may determine the neurotransmitter levels after the drug(s) has taken effect to determine, in conjunction with further QEEG analysis, if a change in a desired direction has taken place and what the magnitude of this change was. Data from the follow-up MRS of the patient 1 before and after administering the drug(s) may be recorded in a database similar to the medicine database 5. As increasing amounts of information is entered relating to different drugs and dosages, a master MRS database may be created that may serve as a reference for the system to properly diagnose an individual based on exhibited MRS activity (step 705). The master MRS database may include information regarding the effects of dosages of specific drugs on the MRS of selected ROIs when the maximum LORETA effects of each drug were found.

Furthermore, the exemplary method of FIG. 7 may also utilize BSVs, wherein an initial BSV may be created for a patient 1 before administering drugs and a subsequent BSV may be taken after administering drugs. A BSV database may thereby be created using the same method as described above with respect to the MRS database.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of determining a pharmacological treatment, comprising:
determining whether a difference exists between brain activity data corresponding to electrical brain activity of a living body and control data corresponding to normative brain activity data;
generating a first brain state vector (BSV) for the living body having an orientation and a length corresponding, respectively, to a quality and a magnitude of any determined difference; and
determining a pharmacological treatment based on at least one of the length and the orientation of the first BSV.

2. The method according to claim 1, wherein the determining of the pharmacological treatment includes:
selecting at least one drug associated with a second BSV orientation that is opposite that of the first BSV.

3. The method according to claim 2, father comprising:
determining a dosage of the drug based on the length of the first BSV.

4. The method according to claim 2, wherein the determining of the pharmacological treatment includes:
selecting a first drug associated with a third BSV orientation and a second drug associated with a fourth BSV orientation so that a vector summation of the third BSV and the fourth BSV produces a resultant BSV that is substantially opposite in orientation with respect to the orientation of the first BSV of the living body.

5. The method according to claim 4, wherein the pharmacological treatment is one of manually administered and automatically administered by a titration unit.

6. The method according to claim 4, further comprising:
monitoring the first BSV after a beginning of the pharmacological treatment to determine whether the first BSV is being reduced to a point of origin of a brain state vector space.

7. The method according to claim 6, further comprising:
if the magnitude of the first BSV one of remains constant and becomes larger after the beginning of the pharmacological treatment, selecting a new pharmacological treatment.

8. A system for determining a pharmacological treatment, comprising:
an arrangement for determining whether a difference exists between brain activity data corresponding to electrical brain activity of a living body and control data corresponding to normative brain activity data;
an arrangement for generating a first brain state vector (BSV) for the living body having an orientation and a length corresponding, respectively, to a quality and a magnitude of any determined difference; and
an arrangement for determining a pharmacological treatment based on at least one of the length and the orientation of the first BSV.

9. The system according to claim 8, wherein the arrangement for determining the pharmacological treatment includes:
an arrangement for selecting at least one drug associated with a second BSV orientation that is opposite that of the first BSV.

10. The system according to claim 8, wherein the arrangement for determining the pharmacological treatment includes:
an arrangement for selecting a first drug associated with a third BSV orientation and a second drug associated with a fourth BSV orientation so that a vector summation of the third BSV and the fourth BSV produces a resultant BSV that is substantially opposite in orientation with respect to the orientation of the first BSV of the living body.

11. The system according to claim 8, further comprising:
an arrangement for monitoring the first BSV after a beginning of the pharmacological treatment to determine whether the first BSV is being reduced to a point of origin of a brain state vector space.

12. The system according to claim 11, further comprising:
an arrangement for, if the magnitude of the first BSV one of remains constant and becomes larger after the beginning of the pharmacological treatment, selecting a new pharmacological treatment.

13. An apparatus for determining a pharmacological treatment for a patient, comprising:
an EEG unit for producing an EEG of the patient;
a QEEG unit for producing patient QEEG data based on the EEG;
a database containing normalized QEEG data derived from one of a population norm and a self-norm;
a processing unit programmed to:
determine whether a difference exists between brain activity data corresponding to electrical brain activity of a living body and control data corresponding to normative brain activity data, generate a first brain state vector (BSV) for the living body having an orientation and a length corresponding, respectively, to a quality and a magnitude of any determined difference, and
determine a pharmacological treatment based on at least one of the length and the orientation of the first BSV.

14. The apparatus of claim 13, wherein the pharmacological treatment exhibits a second BSV orientation that is opposite that of the first BSV.

15. The apparatus of claim 13, further comprising an arrangement automatically indicating the required pharmacological treatment and dosage based on at least one of the length and the orientation of the first BSV.

16. The apparatus of claim 13, further comprising a titration unit connected to a supply of the pharmacological treatment from a pharmacological database to automatically administer the pharmacological treatment.
